(19) United States
(12) Patent Application Publication
(10) Pub. No.: US 2025/0261897 A1
(43) Pub. Date: Aug. 21, 2025

(54) DEMENTIA DIAGNOSIS METHOD AND DEMENTIA DIAGNOSIS SYSTEM USING GENETIC INFORMATION DATA AND BRAIN IMAGE DATA

(71) Applicant: GRADIANT BIOCONVERGENCE INC., Seoul (KR)

(72) Inventors: Si Yun CHUNG, Seoul (KR); Ba Da PYO, Seoul (KR)

(21) Appl. No.: 19/197,443

(22) Filed: May 2, 2025

Related U.S. Application Data
(63) Continuation of application No. PCT/KR2024/005595, filed on Apr. 25, 2024.

(30) Foreign Application Priority Data
May 25, 2023 (KR) 10-2023-0067642

(51) Int. Cl. A61B 5/00; A61B 5/055; G06T 7/00; G16B 25/10; G16H 10/40; G16H 10/60; G16H 30/40; G16H 50/20; G16H 50/70

(52) U.S. Cl. CPC A61B 5/4088; A61B 5/0042; A61B 5/055; G06T 7/0012; G16B 25/10; G16H 10/40; G16H 10/60; G16H 30/40; G16H 50/20; G16H 50/70; A61B 2576/026; G06T 2207/20081; G06T 2207/30016

(57) ABSTRACT

A dementia diagnosis method according to one embodiment includes a step of generating genetic characteristic data by comparing and analyzing genetic information of a normal person and genetic information of a dementia patient; a step of encoding each of brain image data of the normal person and brain image data of the dementia patient, and generating brain feature data for each of the encoded brain image data of the normal person and brain image data of the dementia patient; and a step of generating a dementia diagnosis model trained to determine whether a specific person has dementia based on genetic information and genetic feature data of the specific person using data that combines the genetic feature data and the brain feature data according to a preset method as learning data.

### CROSS REFERENCE TO RELATED APPLICATION

This application is a continuation application of the International Application No. PCT/KR2024/005595, filed on Apr. 25, 2024, which claims priority from Korean Patent Application No. 10-2023-0067642, filed on May 25, 2023, which is also incorporated herein by reference in its entirety.

### TECHNICAL FIELD

The present invention relates to a dementia diagnosis method and a dementia diagnosis system using genetic information data and brain image data, and more specifically, to a dementia diagnosis method and a dementia diagnosis system based on a model learned by analyzing genetic information of a normal person and genetic information of a dementia patient, and brain image data of a normal person and brain image data of a dementia patient, extracting feature data therefrom, and combining the feature data.

### BACKGROUND ART

Dementia is a disease in which an area of the cerebrum or the hippocampus responsible for memory of the brain decreases as beta-amyloid accumulates.

According to the statistics on ‘Dementia Status in Korea’ published by the Central Dementia Center, the number of dementia patients aged 65 or older was 648,223 (prevalence rate of 9.8%) as of 2015, and it is estimated that the number of dementia patients will exceed 1 million (prevalence rate of 10.3%) in 2024, and will exceed 2 million (prevalence rate of 12.3%) in 2041.

In addition, existing dementia examination tools require a long examination time of at least 30 minutes, and mild cognitive impairment or dementia is diagnosed through MRI imaging, memory questionnaire, and doctor's opinion. However, there is a shortage of specialized medical personnel such as dementia specialists, neuropsychologists, or clinical psychologists who have completed specialized training, and there are not enough separate examination sites, so there is a shortage of hospitals that may treat dementia.

Accordingly, a new dementia diagnosis method other than the doctor's opinion is needed.

### DISCLOSURE OF INVENTION

### Technical Problem

The present disclosure is intended to solve the problems of the above-mentioned prior art, and provides a dementia diagnosis method and a dementia diagnosis system based on a model learned by analyzing genetic information of a normal person and genetic information of a dementia patient, and brain image data of a normal person and brain image data of a dementia patient, extracting feature data therefrom, and combining the feature data.

The technical problems to be solved by the present invention are not limited to the technical problems described above, and other technical problems of the present invention may be derived from the following description.

### Solution to Problem

As technical means for solving the above-described technical problem, an embodiment according to a first aspect of the present disclosure provides a dementia diagnosis method. The method includes a step of generating genetic characteristic data by comparing and analyzing genetic information of a normal person and genetic information of a dementia patient; a step of encoding each of brain image data of the normal person and brain image data of the dementia patient, and generating brain feature data for each of the encoded brain image data of the normal person and brain image data of the dementia patient; and a step of generating a dementia diagnosis model trained to determine whether a specific person has dementia based on genetic information and genetic feature data of the specific person using data that combines the genetic feature data and the brain feature data according to a preset method as learning data.

In addition, an embodiment according to a second aspect of the present disclosure provides a dementia diagnosis system. The system includes a communication module; at least one processor; and a memory that is electrically connected to the processor and stores at least one code to be executed by the processor, in which the memory stores a code that, when executed through the processor, causes the processor to generate genetic characteristic data by comparing and analyzing genetic information of a normal person and genetic information of a dementia patient, to encode each of brain image data of the normal person and brain image data of the dementia patient, to generate brain feature data for each of the encoded brain image data of the normal person and brain image data of the dementia patient, and to generate a dementia diagnosis model trained to determine whether a specific person has dementia based on genetic information and genetic feature data of the specific person using data that combines the genetic feature data and the brain feature data according to a preset method as learning data.

### Advantageous Effects of Invention

According to the present invention, accuracy of the diagnosis may be improved by diagnosing the dementia by considering both the genetic data and the brain image data.

In addition, according to the present invention, the convenience of examination may be increased by diagnosing the dementia based on the genetic data and the brain image data.

The effects of the present invention are not limited to the effects described above, and include all effects understood from the following description.

### BRIEF DESCRIPTION OF DRAWINGS

FIG. 1 is a diagram illustrating a dementia diagnosis system according to one embodiment of the present invention.

FIG. 2 is a diagram illustrating a detailed configuration of a server illustrated in FIG. 1.

FIGS. 3 and 4 are diagrams illustrating examples of genetic feature data.

FIGS. 5 to 7 are diagrams illustrating examples of extracting brain feature data.

FIGS. 8A and 8B are diagrams illustrating an accuracy of dementia diagnosis results according to a dementia diagnosis model.

FIG. 9 is a flow chart illustrating a sequence of a dementia diagnosis method according to another embodiment of the present invention.

### BEST MODE FOR CARRYING OUT THE INVENTION

Hereinafter, the present disclosure will be described in detail with reference to the attached drawings. However, the present disclosure may be implemented in various different forms and is not limited to the embodiments described herein. In addition, the attached drawings are only provided to facilitate easy understanding of the embodiments disclosed in the present specification, and the technical ideas disclosed in the present specification are not limited by the attached drawings. All terms, including technical and scientific terms, used herein should be interpreted as having meaning generally understood by a person having ordinary skill in the technical field to which the present disclosure belongs. Terms defined in the dictionary should be interpreted as having additional meaning consistent with the related technical literatures and the contents of the present disclosure, and are not interpreted in a very ideal or restrictive sense unless otherwise defined.

In order to clearly explain the present disclosure in the drawings, parts that are not related to the explanation have been omitted, and the size, shape, and appearance of each component illustrated in the drawings may be modified in various ways. Identical/similar parts throughout the specification are given identical/similar drawing reference numerals.

The suffixes “module” and “portion” used in the following description for components are given or used interchangeably only for the convenience of writing the specification, and do not have distinct meanings or roles in themselves. In addition, when describing the embodiments disclosed in the present specification, if it is determined that a specific description of a related known technology may obscure the gist of the embodiments disclosed in the present specification, the detailed description thereof has been omitted.

Throughout the specification, when a part is said to be “connected (connected, contacted, or coupled)” with another part, this includes not only cases where it is “directly connected (connected, contacted, or coupled)” but also cases where it is “indirectly connected (connected, contacted, or coupled)” with another member therebetween. In addition, when a part is said to “include (have or provide)” a component, this does not exclude other components unless specifically stated to the contrary, but rather means that it may “include (have or provide)” other components.

The terms indicating ordinal numbers such as first, second, etc. used in the present specification are only used for the purpose of distinguishing one component from another component, and do not limit the order or relationship of the components. For example, the first component of the present disclosure may be referred to as the second component, and similarly, the second component may also be referred to as the first component. The singular forms used in the present specification should be construed to include the plural forms as well, unless clearly indicated to the contrary.

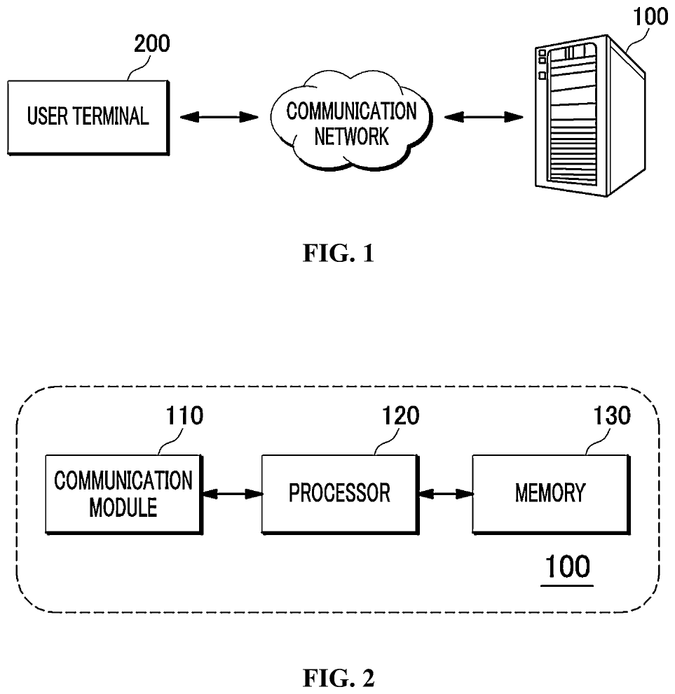

FIG. 1 is a diagram illustrating a dementia diagnosis system according to one embodiment of the present invention.

Referring to FIG. 1, the dementia diagnosis system may include a server 100 and a user terminal 200. The server 100 and the user terminal 200 may be connected to each other through a communication network.

The server 100 compares and analyzes genetic information of a normal person and genetic information of a dementia patient to generate or extract genetic characteristic data.

The server 100 encodes each of brain image data of the normal person and brain image data of the dementia patient. The server 100 generates or extracts brain feature data for each of the coded brain image data of the normal person and brain image data of the dementia patient. For example, the brain image data may be an MRI picture of the brain.

The server 100 uses data that combines the genetic feature data and the brain feature data according to a preset method as learning data, and generates a dementia diagnosis model trained to determine whether a specific person has dementia based on genetic information and genetic feature data of the specific person. The server 100 diagnoses dementia for the input genetic information and brain image data based on the generated dementia diagnosis model.

The user terminal 200 may receive a dementia diagnosis result from the server 100. In addition, the user terminal 200 may transmit the genetic information and the brain image data to the server 100.

The user terminal 200 may be connected to the server 100 through a communication network. The user terminal 200 may mean any type of handheld-based wireless communication device such as a notebook, a desktop, laptop, a wireless communication device with portability and mobility, a smart phone, or a tablet PC equipped with a web browser.

FIG. 2 is a diagram illustrating a detailed configuration of the server illustrated in FIG. 1.

Referring to FIG. 2, the server 100 may include a communication module 110, a processor 120, and a memory 130.

The communication module 110 may include a device including hardware and software necessary to transmit and receive signals such as control signals or data signals, through wired or wireless connections with other network devices.

The communication module 110 may receive the genetic information and the brain image data of the normal person, the mild cognitive impairment patient, and the dementia patient from the user terminal. In addition, the communication module 110 may transmit the dementia diagnosis results to the user terminal.

The processor 120 may include various types of devices that control and process data. The processor 120 may mean a data processing device built into hardware that has a physically structured circuit to perform a function expressed by a code or a command included in a program.

In one example, the processor 120 may be implemented in a form of a microprocessor, a central processing unit (CPU), a processor core, a multiprocessor, an application-specific integrated circuit (ASIC), a field programmable gate array (FPGA), or the like, but the scope of the present invention is not limited thereto.

The processor 120 performs an operation according to the code stored in the memory 130.

The memory 130 may store at least one of information and data input to the communication module 110, information and data required for functions performed by the processor 120, and data generated according to the execution of the processor 120.

The memory 130 should be interpreted as a general term for a nonvolatile storage device that maintains stored information even when power is not supplied and a volatile storage device that requires power to maintain stored information. The memory 130 may include a magnetic storage medium or a flash storage medium in addition to the volatile storage device that requires power to maintain the stored information, but the scope of the present invention is not limited thereto.

The memory 130 is electrically connected to the processor 120 and stores at least one code that is executed in the processor 120. The memory 130 stores a code that causes the processor 120 to perform the following functions and procedures when the code is executed through the processor 120.

The memory 130 stores the code that causes the genetic feature data to be generated by comparing and analyzing the genetic information of the normal person and the genetic information of the dementia patient. For example, the genetic information may be data on the abundance of bacteria for each sample through analysis of 16s rRNA sequencing data for a fecal sample, which may be data on a composition of the total microorganisms. In addition, the genetic feature data may be a vector generated based on the number of occurrences of each bacteria.

The memory 130 may store a code that causes bacterial data to be generated by clustering bacterial information of each of the genetic information of the normal person and the genetic information of the dementia patient. The memory 130 may store a code that causes the generated bacterial data to be converted into numerical data to extract vector-type genetic feature data. For example, the memory 130 may perform ASV clustering and classification annotation (Taxon annotation) on the 16s rRNA data based on the stored code.

For example, the memory 130 may convert ASV data into numerical data based on the stored code and perform one-hot encoding on each ASV sample to generate a numerical vector representing ASV characteristics for each sample. Here, the ASV may be taxonomic information matched to a species.

In addition, the memory 130 may store a code that causes the genetic information of the mild cognitive impairment patient to be compared and analyzed to extract the genetic feature data.

In addition, the memory 130 may store a code that causes the features for the brain image data to be extracted using CNN and the brain image data for each of the normal person, the mild cognitive impairment patient, and the dementia patient to be vectorized.

The memory 130 stores a code that causes each of the brain image data of the normal person and the brain image data of the dementia patient to be encoded. For example, the memory 130 may store a code that causes each of the brain image data of the normal person and the brain image data of the dementia patient to be encoded for each region. Here, the brain image data may be a brain MRI image, and the memory 130 may store a code that causes the brain MRI picture to be data into the brain image data based on the dementia diagnosis model.

The memory 130 stores a code that causes the brain feature data to be generated for each of the coded brain image data of the normal person and brain image data of the dementia patient. In addition, the memory 130 may store a code that causes the brain feature data for the brain image data of the mild cognitive impairment patient to be extracted.

For example, the memory 130 may store a code that causes the brain feature data of a vector form to be generated based on each of the brain image data of the normal person, the brain image data of the dementia patient, and the brain image data of the mild cognitive impairment patient, and a preset feature detection method.

The memory 130 stores a code that causes the dementia diagnosis model that is trained to determine whether a specific person has dementia based on the genetic information and genetic feature data of the specific person, using data that combines the genetic feature data, the brain feature data of the normal person, and the brain feature data of the dementia patient according to a preset method as learning data. For example, the memory 130 may store a code that causes a single input vector to be generated by connecting the genetic feature data and the brain feature data. For example, the memory 130 may generate the single input vector by connecting the brain feature data acquired from the CNN and the converted genetic feature data based on the stored code.

The memory 130 may store a code that causes the dementia diagnosis model to be generated by learning the generated input vector. For example, the memory 130 may input the combined input vector into the dementia diagnosis model based on the stored code and learn and perform the dementia diagnosis model. The memory 130 may produce a prediction value and loss for dementia or normal. Here, the dementia diagnosis model may be a model generated using Keras.

The memory 130 stores a code that causes dementia to be diagnosed based on the input genetic information and brain image data based on the dementia diagnosis model. For example, the memory 130 stores a code that causes the genetic information and brain image data of a specific person to be input using the dementia diagnosis model and the state of the specific person to be determined as at least one of mild cognitive impairment, dementia, and normal.

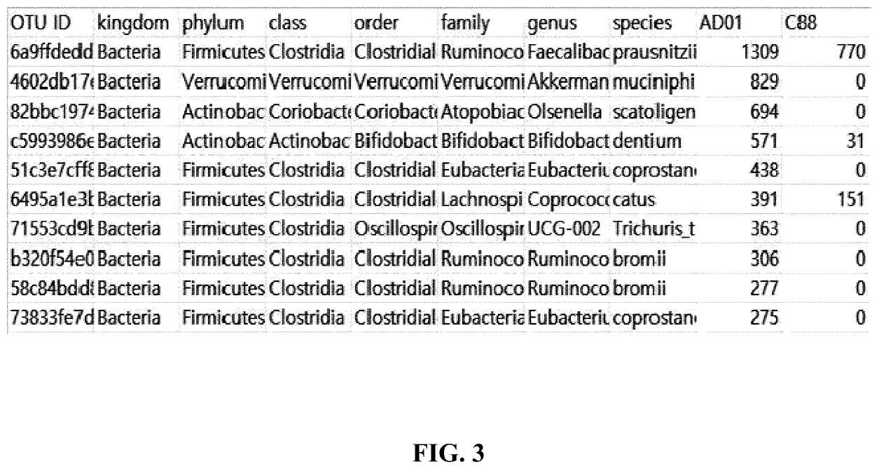

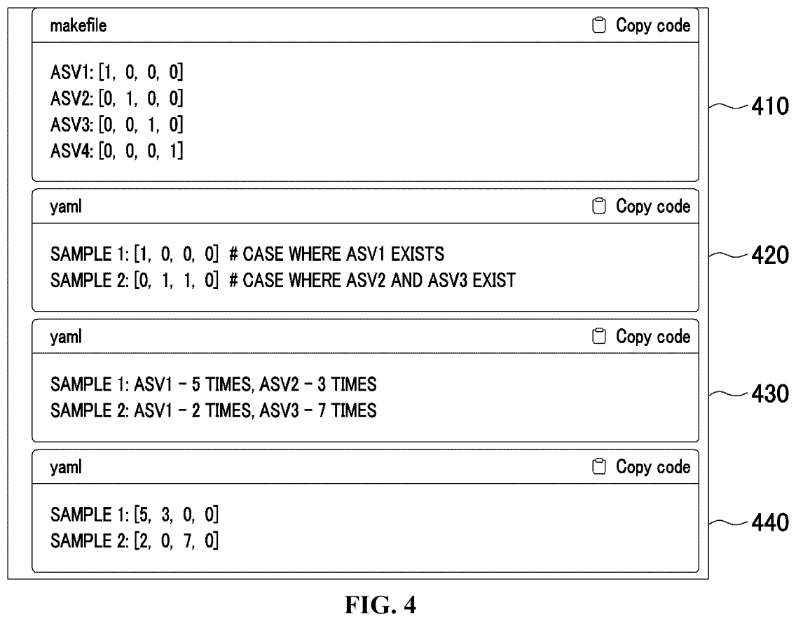

FIGS. 3 and 4 are diagrams illustrating examples of genetic feature data.

Referring to FIGS. 3 and 4, the genetic feature data may include ASV ID for each bacteria, taxonomic information data for ASV, and bacterial information for each sample with respect to samples of the normal person and the dementia patient. For example, the genetic feature data may include information on OUT ID, kingdom, phylum, class, order, family, genus, species, AD01, and C88.

The genetic feature data may be extracted in the form of a vector based on the number of occurrences for each ASV, such as AD1=[1,0,0,1] and C88=[0,1,1,0].

The dementia diagnosis system may convert each ASV into a vector 410 in which each feature has a unique value. For example, if there are four ASVs, the dementia diagnosis system may perform one-hot encoding such as ASV1: [1,0,0,0], ASV2: [0,1,0,0], ASV3: [0,0,1,0], and ASV4: [0,0,0,1].

The dementia diagnosis system may generate transformation data 420 for the sample. For example, the dementia diagnosis system may generate transformation data for sample 1 in which ASV1 exists and sample 2 in which ASVs 2 and 3 exist, such as sample 1: [1,0,0,0], sample 2: [0,1,1,0].

The dementia diagnosis system may extract genetic feature data 440 in vector form based on the number of occurrences 430 of each ASV. For example, if ASV1 appears 5 times and ASV3 appears 3 times in sample 1, and ASV1 appears 2 times and ASV3 appears 7 times in sample 2, the dementia diagnosis system may extract genetic feature data 440 such as sample 1: [5,3,0,0] and sample 2: [2,0,7,0].

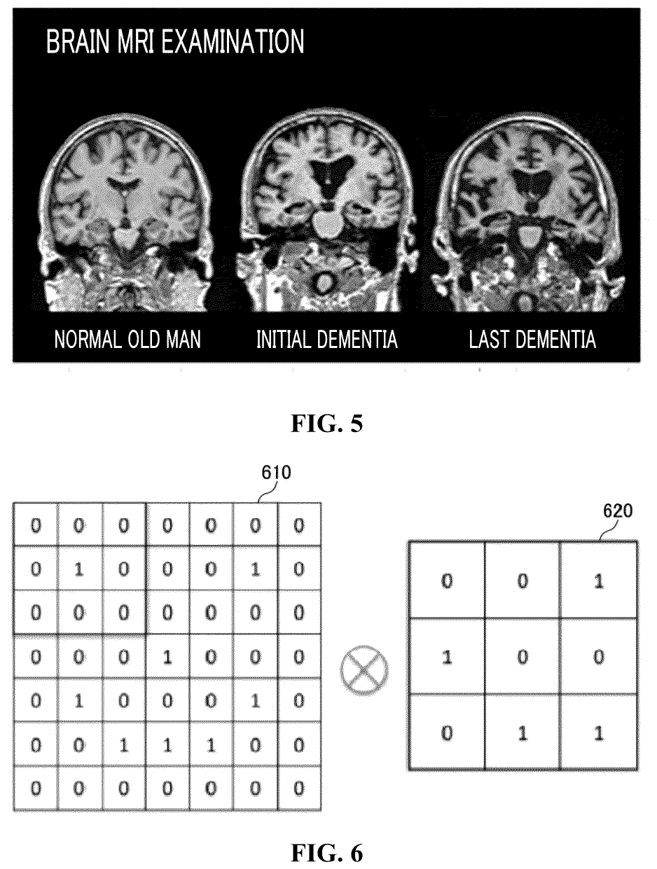

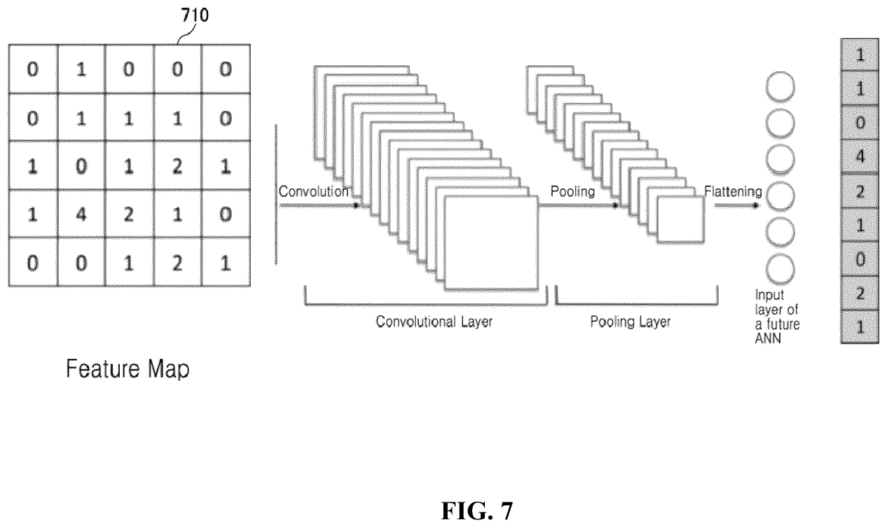

FIGS. 5 to 7 are diagrams illustrating examples of extracting brain feature data.

Referring to FIGS. 5 to 7, the dementia diagnosis system may code each region of the MRI picture of each of the normal person, the dementia patient, and the mild cognitive impairment patient. The dementia diagnosis system may generate a feature map 710 by combining brain image data 610 and feature detection data 620 which are coded for each region.

The dementia diagnosis system may extract brain feature data of the vector form by repeatedly performing Max pooling and flattening on the feature map 710 to create a layer for the features of the brain image data. The dementia diagnosis system may diagnose the dementia by analyzing newly input brain image data and genetic information based on the brain feature data.

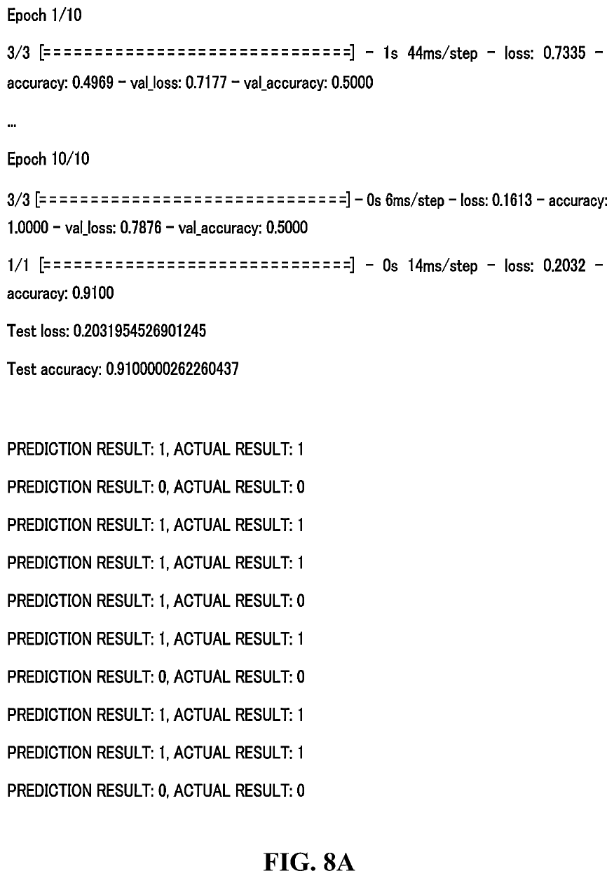

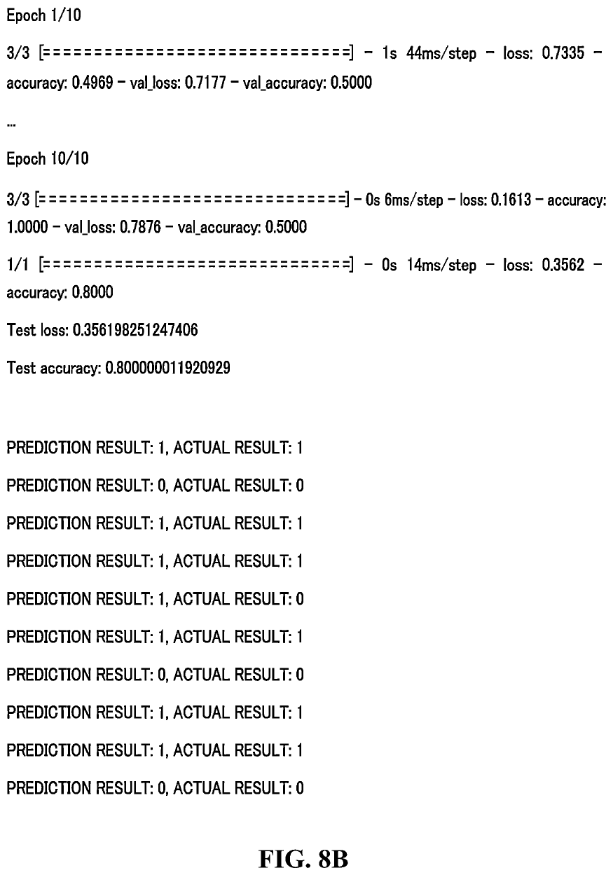

FIGS. 8A and 8B are diagrams illustrating an accuracy of dementia diagnosis results according to the dementia diagnosis model.

Referring to FIG. 8A, it is confirmed that the dementia diagnosis model is created by combining the MRI picture and the 16s rRNA data, and the results of diagnosing dementia illustrate that the loss for the test set is 0.203 and the accuracy is 0.91. In this case, the dementia diagnosis model may be learned for 10 epochs.

On the other hand, referring to FIG. 8B, it is confirmed that the dementia diagnosis model is created only by the 16s rRNA data without MRI picture, and the results of diagnosing dementia illustrate that the loss for the test set is 0.356 and the accuracy is 0.80.

Accordingly, when the dementia diagnosis model is created by combining the MRI picture and the 16s rRNA data, and dementia is diagnosed, it is confirmed that the accuracy increases to 0.91 and the loss is reduced by 0.153 compared to when the dementia diagnosis model is created and dementia is diagnosed using only 16s rRNA data.

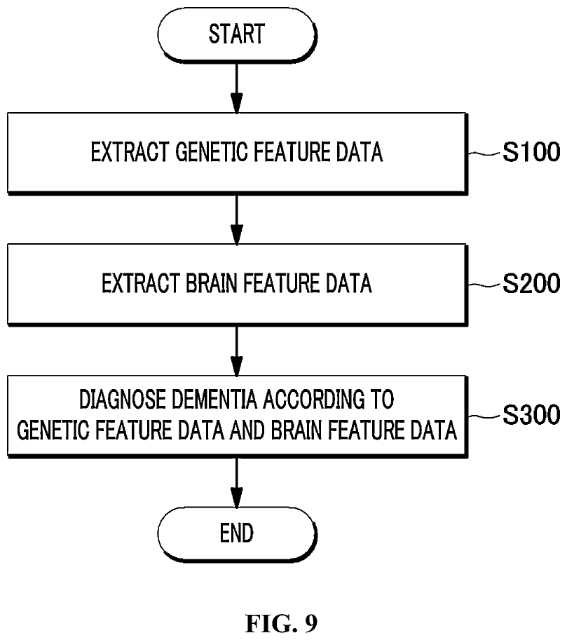

FIG. 9 is a flow chart illustrating a sequence of a dementia diagnosis method according to another embodiment of the present invention.

The dementia diagnosis method described below may be performed by the dementia diagnosis system and the server described above with reference to FIGS. 1 to 8. Therefore, the contents of the embodiments of the present disclosure described above with reference to FIGS. 1 to 8 may be equally applied to the embodiments described below, and any duplicated contents with the above-described description will be omitted. The steps described below do not necessarily have to be performed in order, and the order of the steps may be set in various ways, and the steps may be performed almost simultaneously.

Referring to FIG. 9, the dementia diagnosis method includes a genetic feature data extraction step (S100), a brain feature data extraction step (S200), and a dementia diagnosis step (S300) according to the genetic feature data and brain feature data.

The genetic feature data extraction step (S100) is a step of extracting the genetic feature data by comparing and analyzing the genetic information of the normal person and the genetic information of the dementia patient. For example, in the genetic feature data extraction step (S100), bacterial information of each of the genetic information of the normal person and the genetic information of the dementia patient may be clustered to generate bacterial data, and the bacterial data may be converted into numerical data to extract the genetic feature data of the vector form.

The brain feature data extraction step (S200) is a step of encoding each of the brain image data of the normal person and the brain image data of the dementia patient, and extracting the brain feature data for each of the encoded brain image data of the normal person and brain image data of the dementia patient. For example, in the brain feature data extraction step (S200), each of the brain image data of the normal person and the brain image data of the dementia patient may be encoded for each region, and the brain feature data of the vector form may be extracted based on the encoded brain image data and the preset feature detection data.

The dementia diagnosis step (S300) according to the genetic feature data and the brain feature data is a step of generating the dementia diagnosis model trained to determine whether a specific person has dementia based on the genetic information and the genetic feature data of the specific person using data that combines the genetic feature data and the brain feature data according to a preset method as learning data. For example, in the dementia diagnosis step (S300) according to the genetic feature data and brain feature data, one input vector is generated by connecting the genetic feature data and the brain feature data, the dementia diagnosis model is learned using the input vector, and dementia may be diagnosed for the input genetic information and brain image data based on the dementia diagnosis model.

Those skilled in the art will appreciate that the present disclosure may be easily modified into other specific forms without changing the technical idea or essential features of the present disclosure based on the above description. Therefore, it should be understood that the embodiments described above are exemplary in all respects and not restrictive. The scope of the present disclosure is indicated by the claims below, and all changes or modifications derived from the meaning and scope of the claims and their equivalents should be interpreted as being included in the scope of the present disclosure. The scope of the present application is indicated by the claims below rather than the detailed description above, and all changes or modifications derived from the meaning and scope of the claims and their equivalents should be interpreted as being included in the scope of the present application.

### INDUSTRIAL APPLICABILITY

Since the present invention may be used in a technology for diagnosing dementia, it has industrial applicability.

1. A dementia diagnosis method performed by a dementia diagnosis system, the dementia diagnosis method comprising:
(a) a step of generating genetic characteristic data by comparing and analyzing genetic information of a normal person and genetic information of a dementia patient;
(b) a step of encoding each of brain image data of the normal person and brain image data of the dementia patient, and generating brain feature data for each of the encoded brain image data of the normal person and brain image data of the dementia patient; and
(c) a step of generating a dementia diagnosis model trained to determine whether a specific person has dementia based on genetic information and genetic feature data of the specific person using data that combines the genetic feature data and the brain feature data according to a preset method as learning data.

2. The dementia diagnosis method of claim 1, wherein
in the step (a),
bacterial data is generated by clustering bacterial information of each of the genetic information of the normal person and the genetic information of the dementia patient, and the genetic feature data of a vector form is generated by converting the bacterial data into numerical data.

3. The dementia diagnosis method of claim 2, wherein
the genetic feature data is vector data generated based on the number of occurrences of each bacteria.

4. The dementia diagnosis method of claim 1, wherein
the genetic information includes 16s rRNA data.

5. The dementia diagnosis method of claim 1, wherein
in step (b),
each of the brain image data of the normal person and the brain image data of the dementia patient is encoded for each region, and the brain feature data of a vector form is generated based on the encoded brain image data and a preset feature detection method.

6. The dementia diagnosis method of claim 1, wherein
in step (c),
the genetic feature data and the brain feature data are connected to generate one input vector, the dementia diagnosis model is learned based on the input vector.

7. The dementia diagnosis method of claim 1, wherein
the step (a) includes a step of generating the genetic feature data by comparing and analyzing genetic information of a mild cognitive impairment patient and the genetic information of the normal person, and
the step (b) includes a step of generating the brain feature data by comparing and analyzing brain image data of the mild cognitive impairment patient and the brain image data of the normal person.

8. The dementia diagnosis method of claim 1, wherein
the step (c) includes a step of inputting genetic information and brain image data of a specific person using the dementia diagnosis model and determining a state of the specific person as at least one of mild cognitive impairment, dementia, and normal.

9. A dementia diagnosis system comprising:
a communication module;
at least one processor; and
a memory that is electrically connected to the processor and stores at least one code to be executed by the processor,
wherein the memory stores a code that, when executed through the processor, causes the processor to generate genetic characteristic data by comparing and analyzing genetic information of a normal person and genetic information of a dementia patient, to encode each of brain image data of the normal person and brain image data of the dementia patient, to generate brain feature data for each of the encoded brain image data of the normal person and brain image data of the dementia patient, and to generate a dementia diagnosis model trained to determine whether a specific person has dementia based on genetic information and genetic feature data of the specific person using data that combines the genetic feature data and the brain feature data according to a preset method as learning data.

10. The dementia diagnosis system of claim 9, wherein
the memory a code that causes the processor to generate bacterial data by clustering bacterial information of each of the genetic information of the normal person and the genetic information of the dementia patient, and to extract the genetic feature data of a vector form by converting the bacterial data into numerical data.

11. The dementia diagnosis system of claim 9, wherein
the genetic feature data is vector data generated based on the number of appearances for each bacteria.

12. The dementia diagnosis system of claim 9, wherein
the memory stores a code that causes the processor to encode each of the brain image data of the normal person and the brain image data of the dementia patient for each region, and to extract the brain feature data of a vector form based on the encoded brain image data and the preset feature detection data.

13. The dementia diagnosis system of claim 9, wherein
the memory stores a code that causes the processor to generate one input vector by connecting the genetic feature data and the brain feature data, to learn the dementia diagnosis model using the input vector, and to diagnose the dementia based on the dementia diagnosis model.

14. The dementia diagnosis system of claim 9, wherein
the memory stores a code that causes the processor to generate the genetic feature data by comparing and analyzing genetic information of a mild cognitive impairment patient and the genetic information of the normal person, and to generate the brain feature data by comparing and analyzing the brain image data of the mild cognitive impairment patient and the brain image data of the normal person.

15. The dementia diagnosis system of claim 9, wherein
the memory stores a code that causes the processor to input the genetic information and brain image data of a specific person using the dementia diagnosis model, and to determine a state of the specific person as at least one of mild cognitive impairment, dementia, and normal.
